(19) United States
(12) Patent Application Publication
(10) Pub. No.: US 2012/0010506 A1
(43) Pub. Date: Jan. 12, 2012

(54) MULTIMODAL LAPAROSCOPIC ULTRASOUND DEVICE WITH FEEDBACK SYSTEM

(75) Inventor: Christopher J. Ullrich, Ventura, CA (US)

(73) Assignee: IMMERSION CORPORATION, San Jose, CA (US)

(21) Appl. No.: 12/832,630

(22) Filed: Jul. 8, 2010

(51) Int. Cl. A61B 8/00

(52) U.S. Cl. 600/440; 600/437

(57) ABSTRACT

A laparoscopic tool or minimally invasive device is augmented with a forward looking ultrasonic transducer that is processed to extract information regarding subsurface structures and to generate haptic, audio, or visual effects to provide relevant feedback to a user that is operating the tool. In one embodiment, the ultrasonic transducer detects the distance or depth of subsurface structures such as a luminal hollow structure or a tumor mass. In another embodiment, the ultrasonic transducer extracts tissue identification information, tissue stiffness, velocity, or other pertinent information regarding subsurface structures that is subsequently communicated to the operator as haptic, audio, and/or visual feedback. The ultrasonic transducer may be operable in one or more modes, including A-mode or Doppler mode.

### FIELD OF THE INVENTION

Embodiments hereof relate to surgical tools having an ultrasound transducer at a distal end for detecting information about subsurface structures that is processed and displayed to a user in one or more feedback modes integrated onto a handle of the tool.

### BACKGROUND OF THE INVENTION

As opposed to open surgery in which a surgeon cuts a relatively large incision in the skin of a patient for accessing internal organs, minimally invasive surgical procedures are performed by making relatively small incisions and then inserting tools through the incisions to access the organs. Minimally invasive surgery usually results in shorter hospitalization times, reduced therapy requirements, less pain, less scarring, and fewer complications.

Although minimally invasive surgical procedures involving small incisions include many advantages over open surgery, minimally invasive surgery can still create challenges to a surgeon. For example, the surgeon must typically rely on a miniature camera introduced through an incision to view the patient's internal organs and see how the movement and operation of the tools affects the organs. The camera transmits images to a visual display, allowing the surgeon to see the internal organs and tissues and to see the effect of other minimally invasive tools on the organs and tissues. In this way, the surgeon is able to perform laparoscopic surgery, dissection, cauterization, endoscopy, telesurgery, and the like. Compared to open surgery, however, minimally invasive surgery can present limitations regarding the surgeon's ability to see and feel the patient's organs and tissues. In laparoscopic surgery, surgeons have limited capacity to palpate or manipulate tissues to enable procedural progress. In some cases, this can result in converting the procedure from closed to open to facilitate identification of various structures or management of operative complications.

During a minimally invasive procedure, identification/detection of subsurface structures may in some cases avoid the necessity of converting a closed procedure to open. For example, during gynecologic or colorectal surgery it would be desirable to detect the ureter of a patient to avoid inadvertently cutting or transecting the ureter with the laparoscopy tool. In human anatomy, ureters are hollow muscular tubes that propel urine from the kidneys to the urinary bladder. Since the ureter does not contain blood and does not have a pulse, such an injury may go unknown until complications arise around one or two weeks after the gynecologic or colorectal surgery. Such complications, including kidney failure and abdominal infection, may be serious and life-threatening. It is therefore an object of the present invention to provide a surgical laparoscopic or minimally invasive tool that provides haptic, audio, and/or visual feedback to the surgeon about the distance/depth of subsurface luminal structures. In addition, it is an object of the present invention to provide a surgical laparoscopy or minimally invasive tool that provides haptic, audio, and/or visual feedback to the surgeon about other pertinent information including tissue identification, tissue stiffness, velocity information, and the like that will enable the surgeon to feel his or her way through the subsurface tissues.

### BRIEF SUMMARY OF THE INVENTION

Embodiments hereof relate a surgical tool including a handle and a distal portion connected to the handle via a shaft. A forward-looking ultrasonic transducer is mounted on the distal portion, the ultrasonic transducer operable in A-mode and configured to detect information about a subsurface structure. A feedback system is coupled to the handle and configured to communicate the detected information of the subsurface structure to a user.

In addition, embodiments hereof relate to a method of providing feedback to a user during a surgical procedure. In one embodiment, information about a subsurface structure is detected with a forward-looking ultrasonic transducer mounted on a distal portion of a surgical tool, the ultrasonic transducer operating in A-mode. The detected information is processed and communicated to a user via a feedback system coupled to a handle of the surgical tool.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a diagram illustrating a side view of a surgical tool with an ultrasonic transducer mounted at a distal end, according to an embodiment hereof.

FIG. 2 is a block diagram of the ultrasonic transducer, the pulse generator and receiver, and the haptic feedback system of the surgical tool of FIG. 1.

FIG. 3 is a diagram illustrating a side view of a surgical tool having haptic and audio feedback mechanisms, according to an embodiment hereof.

FIG. 4 is a diagram illustrating a side view of a surgical tool having haptic, audio, and visual feedback mechanisms, according to another embodiment hereof.

FIG. 5 is a block diagram of an ultrasonic transducer that operates in A-mode, according to an embodiment hereof.

FIG. 5A is a representative diagram of a A-mode sonogram.

FIG. 6 is a diagram illustrating a side view of a surgical tool with an ultrasonic transducer and a sensor mounted at a distal portion, according to an embodiment hereof.

FIG. 7 is a block diagram of an ultrasonic transducer that operates in A-mode or Doppler mode, according to another embodiment hereof.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

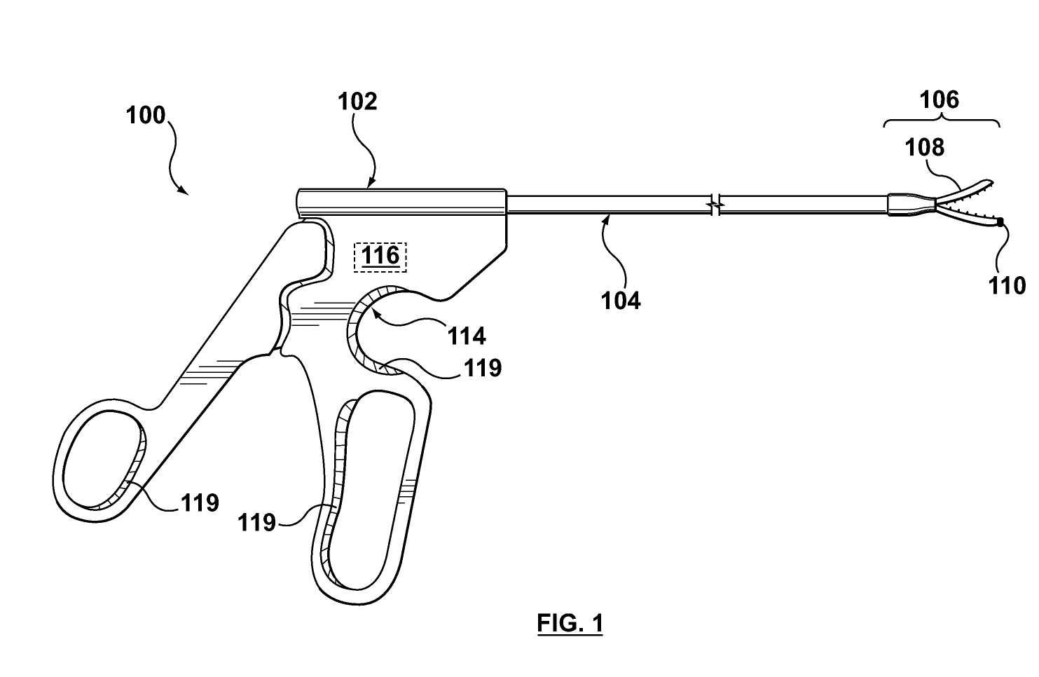

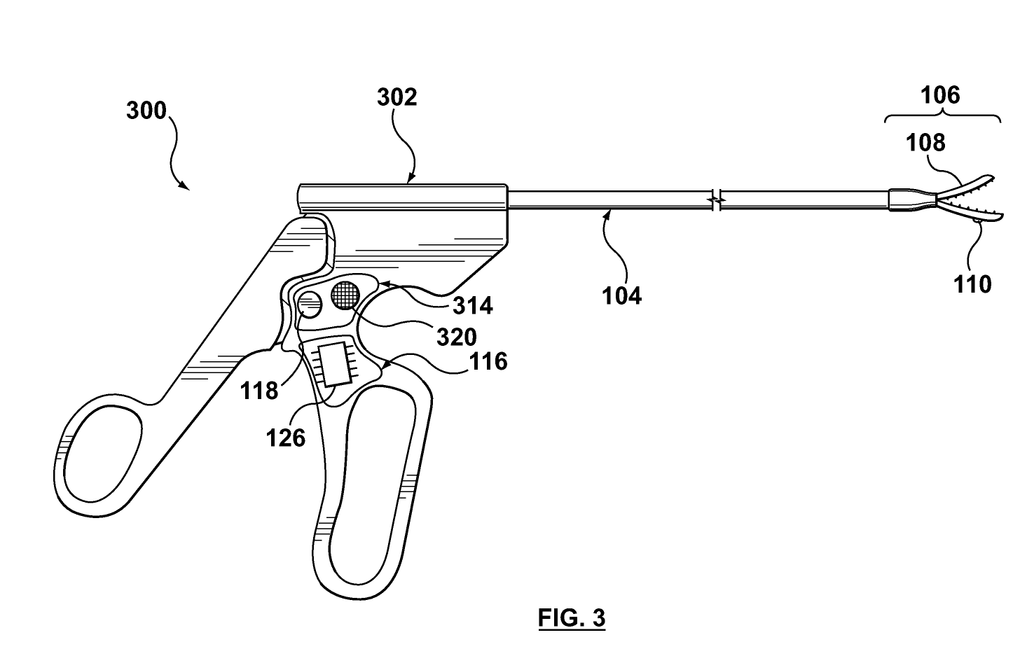

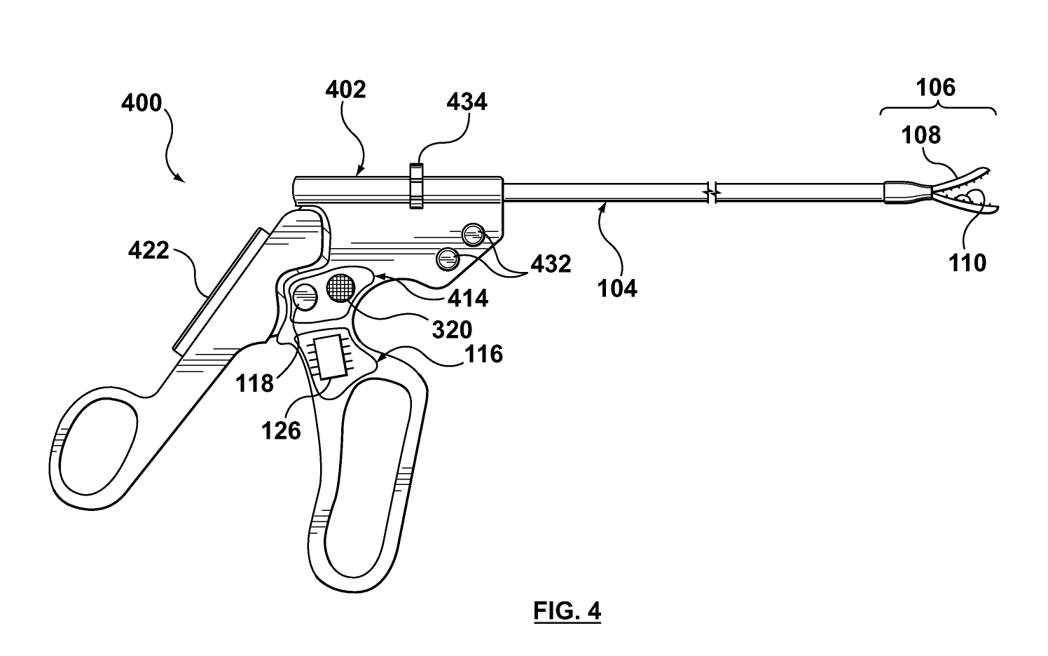

FIG. 1 illustrates an embodiment of a surgical tool 100. In this embodiment, surgical tool 100 is shown as a laparoscopic tool which is configured to be inserted through trocar or other minimally invasive access port. Surgical tool 100 includes a handle 102, a shaft 104, and a distal portion 106. Distal portion 106 includes a tip 108 and an end or forward-looking ultrasonic transducer 110 coupled to and mechanically integrated with tip 108. As shown, tip 108 is a grasper or gripper. However, it should be understood that distal portion 106 may include any suitable type of tip having any suitable functionality. Transducer 110 may be coupled to the distal end of the grasper as shown in FIG. 1, to the bottom or outer surface of the grasper as shown in FIG. 3, to an interior surface within jaws of the grasper as shown in FIG. 4, or any other location on the grasper that is deemed appropriate. Laparoscopic tools in general, like tool 100, are typically thin instruments that each have varied functions (e.g., grippers/graspers, scissors, clip appliers, etc.) and that can be introduced by the surgeon into the abdomen or other areas of the body through trocars, which are hollow tubes with a rubber seal to keep CO₂ from leaking Also, in other alternative embodiments, ultrasonic transducer 110 may be connected to any part of distal portion 106 or other parts of tip 108. Shaft 104 is designed to connect handle 102 to distal portion 106 and to communicate mechanical actions of handle 102 to distal portion 106. More particularly, the motion of handle 102 opens and closes grasper tip 108 through an internal mechanical connector (not shown) that runs from handle 102 to grasper tip 108. According to some examples of the embodiment of FIG. 1, shaft 104 may be about 20 cm to 30 cm in length and tip 108 may be about 10 mm to 15 mm in length. In addition the shaft 104 is typically 5 mm in diameter, although tools with 3 mm, 10 mm and 12 mm diameters are also commonly used. By manipulating handle 102, an operator can insert distal portion 106 into the abdomen of the patient and control tip 108 of distal portion 106. When distal portion 106 is inserted, the surgeon can further manipulate handle 102 to control the location and orientation of tip 108 such that ultrasound transducer 110 is able to contact certain regions of the patient. In one embodiment, ultrasonic transducer 110 is a piezoelectric transducer.

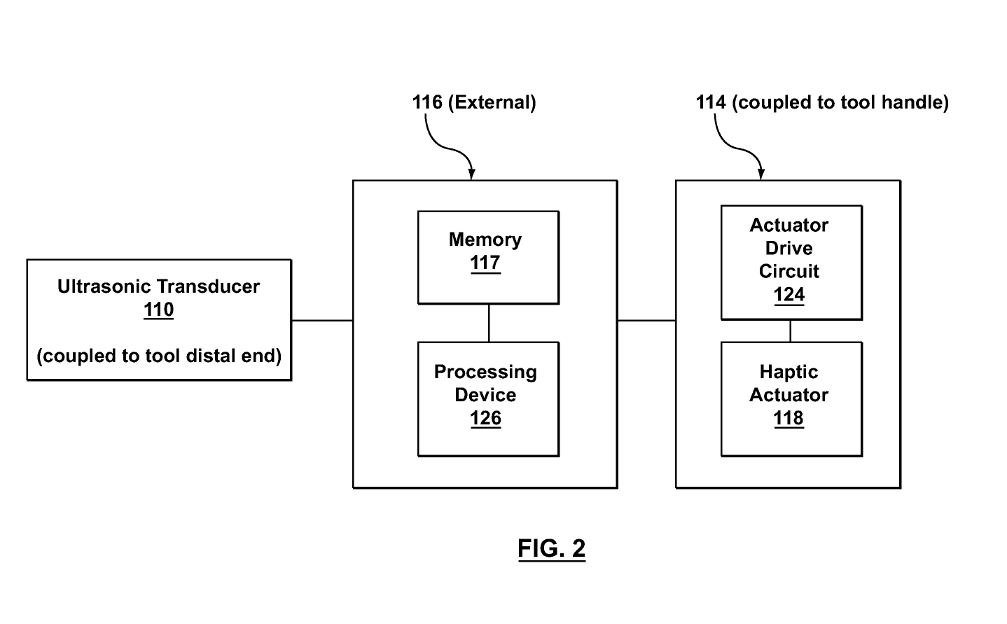

Ultrasonic transducer 110 is electrically connected to a pulse generator and receiver 116. Pulse generator and receiver 116 is capable of generating ultrasonic pulses at sufficient frequency, typically between 2 and 15 Mhz, to penetrate thin layers of tissue approximately 2-5 cm deep. Pulse generator and receiver 116 includes a processing device 126 that receives and processes signals from ultrasonic transducer 110 and communicates the processed information to a haptic feedback system 114 coupled to handle 102 of tool 100, as will be explained in more detail herein. FIG. 2 is a block diagram of ultrasonic transducer 110, pulse generator and receiver 116, and haptic feedback system 114 in accordance with one embodiment hereof. Processing device 126 is configured to process the information detected from ultrasonic transducer 110 according to specific algorithms and operator selections. Depending on the type of properties being measured, the algorithms of processing device 126 can determine various characteristics, properties, or other pertinent information of subsurface tissue structures.

Processing device 126 may be a general-purpose or specific-purpose processing device or microcontroller for processing signals detected by ultrasonic transducer 110. In some embodiments, processing device 126 may include a plurality of processing devices for performing different functions with respect to haptic feedback system 114. In one embodiment, processing device 126 may be associated with a memory device 117 for storing data and/or instructions. Memory device 117 can be any type of storage device or computer-readable medium, such as random access memory (“RAM”) or read-only memory (“ROM”). Memory device 117 stores logical instructions, commands, and/or code executed by processing device 126. Memory device 117 may also be located internal to processing device 126, or any combination of internal and external memory. In another embodiment, logical instructions, commands, and/or code can be implemented in hardware and incorporated in processing device 126 using discrete logic circuitry, an application specific integrated circuit (“ASIC”), a programmable gate array (“PGA”), a field programmable gate array (“FPGA”), etc., or any combination thereof. In yet another embodiment, logical instructions, commands, and/or code can be implemented in both hardware in processing device 126 and software/firmware stored in the memory.

In one embodiment, pulse generator and receiver 116 is an external component or separated from surgical tool 100 and is electrically connected to ultrasound transducer 110 and haptic feedback system 114 via internal or external wires. Pulse generator and receiver 116 is externally powered, and processing device 126 and memory 117 are generally in the same instrumentation unit as pulse generator and receiver 116. In another embodiment, pulse generator and receiver 116 may be mounted within or on handle 102 of surgical tool 100 and one or more internal wires (not shown) extend within shaft 104 of tool 100 to electrically connect pulse generator and receiver 116 to ultrasound transducer 110.

Tool 100 further includes haptic feedback system 114 coupled to handle 102 to provide feedback information to an operator when performing a procedure. Haptic feedback system 114 includes at least an actuator drive circuit 124 which is coupled to a haptic actuator 118 for providing haptic feedback to the operator. In order to provide feedback to the operator, haptic feedback system 114 is electrically connected to pulse generator and receiver 116. Processing device 126 communicates the processed information to haptic actuator 118. Processing device 126 outputs control signals to drive circuit 124 (shown in FIG. 2) which includes electronic components and circuitry used to supply haptic actuator 118 with the required electrical current and voltage to cause the desired haptic effects. In one embodiment, haptic actuator 118 is a vibrotactile device that generates vibrations on handle 102 for haptic feedback. Other types of haptic feedback may be generated and provided to the user, including kinesthetic feedback (e.g., active and resistive force feedback), handle deformation, and/or other types of tactile feedback such as texture and heat. As shown in FIG. 1, multiple haptic actuators may be incorporated in handle 102 at several locations 119 for providing haptic effects to the fingers and thumb of a hand of the surgeon. Haptic actuators 118 may include electromagnetic motors, eccentric rotating mass (“ERM”) actuators in which an eccentric mass is moved by a motor, linear resonant actuators (“LRAs”) in which a mass attached to a spring is driven back and forth, shape memory alloys, electro-active polymers that deform in response to signals, mechanisms for changing stiffness, vibrotactile actuators, inertial actuators, piezoelectric actuators, or other suitable types of actuating devices. In one embodiment, haptic actuator 118 can be implemented as an inertial actuator to provide vibrotactile feedback to the operator. A detailed description of possible haptic actuators suitable for use herein may be found in U.S. Published Patent Application Publication No. 2009/0088659 (application Ser. No. 11/862,639, filed Sep. 27, 2007), herein incorporated by reference in its entirety.

Referring to FIG. 3, an embodiment incorporating two types or modes of feedback for the operator is shown. Specifically, haptic feedback system 314 provides both haptic and audio feedback via haptic actuator 118 and an audio device or speaker 320. Processing device 126 communicates the processed information to one or more of haptic actuator 118 and audio device 320 according to which ones of these feedback mechanisms are enabled and how they are controlled to provide their respective outputs. Pulse generator and receiver 116 including processing device 126, haptic actuator 118, and speaker 320 can be mounted on a printed circuit board (not shown), which includes circuitry for electrically coupling the components. The circuitry on the printed circuit board may include any suitable amplification and attenuation type circuitry, power sources for providing and regulating power to each component, and other circuitry for proper operation of processing device 126 and other components as would be understood by one of ordinary skill in the art.

In one embodiment, feedback may be provided to the operator in a continuous manner as the operator performs the surgery. In another embodiment, feedback may be provided to the operator as an alert to notify or warn the operator when a particular condition is satisfied. Further, one type of feedback may be provided in a continuous manner while another type of feedback is provided as an alert. For example, audio feedback may be provided to the operator in a continuous manner while haptic feedback is provided to the operator as an alert. When haptic feedback system 114 is being utilized to provide the operator with distance or depth feedback as will be explained in more detail herein, continuous audio feedback may inform the operator of the distance remaining between an obscured structure (such as a ureter) and the tip of surgical tool 100 and an haptic alert may be generated when the tip is almost adjacent to the structure.

Referring to FIG. 4, an embodiment incorporating three types or modes of feedback for the operator is shown. Specifically, haptic feedback system 414 provides haptic, audio, and visual feedback via haptic actuator 118, audio device or speaker 320, and visual display 422, respectively. Processing device 126 communicates the processed information to one or more of haptic actuator 118, audio device 320, visual display 422 according to which ones of these feedback mechanisms are enabled and how they are controlled to provide their respective outputs. In this embodiment, visual display 422 is a liquid crystal display (LCD) screen on a back region of handle 102. Visual display 422 may be used to display information regarding the object sensed at a distal end of surgical tool 100. Visual display 422 may be configured to show ultrasound image information to assist the surgeon to position the tool as needed. Visual display 422 can include a touch screen, which can be configured to present information to the operator and can also be configured to sense when the operator presses certain portions of the touch screen. In this way, the touch screen can act as a touchable user interface with graphical presentation capabilities. Visual display 422 may include a graphical user device that enables the surgeon to select different feedback profiles, adjust sensor behavior, modify supplemental information, and the like.

According to the embodiment of FIG. 4, handle portion 402 of surgical tool 400 may further include one or more buttons 432. Buttons 432 can be configured using any suitable mechanism for allowing an operator to control the nature of the feedback that is provided to the operator. Buttons 432 may include devices for allowing certain levels, intensities, or amplitudes to be adjusted or certain selections to be made regarding the output presented to the operator. In some embodiments, buttons 432 may be configured as switches, such as momentary toggle switches, allowing an operator to select different ways in which sensor information is mapped or provided to respective output devices. Buttons 432 can be implemented as a rocker switch or as a one-dimensional control surface. According to one function of buttons 432, the operator can enable or disable one or more output mechanisms by controlling whether or not output signals based on the sensed signals are provided to the respective output devices. Another function of buttons 432 includes the ability to enable one or more output mechanisms. In this regard, the operator can control if and how feedback is presented in a visual, auditory, and/or haptic fashion. With feedback tailored to the surgeon's preferences, the tool can provide feedback to supplement the operator experience for better operation and performance.

Handle 402 may further include a rotary device 434 to be used as a “roll” control device. Rotary device 434 is connected to shaft 104 and, when rotated by operator, is configured to rotate shaft 104 about its axis. Likewise, ultrasound transducer 110 or other sensor connected to a distal portion of surgical tool 100 rotates in response to rotation of rotary device 434. In this way, the operator can control the positioning of ultrasound transducer 110 or other sensor to pick up various signals from different regions of the patient. Although only depicted on FIG. 4, it should be understood to those of ordinary skill in the art that buttons 432 and rotary device 434 may be incorporated into any embodiment described herein for selecting and controlling modes of feedback.

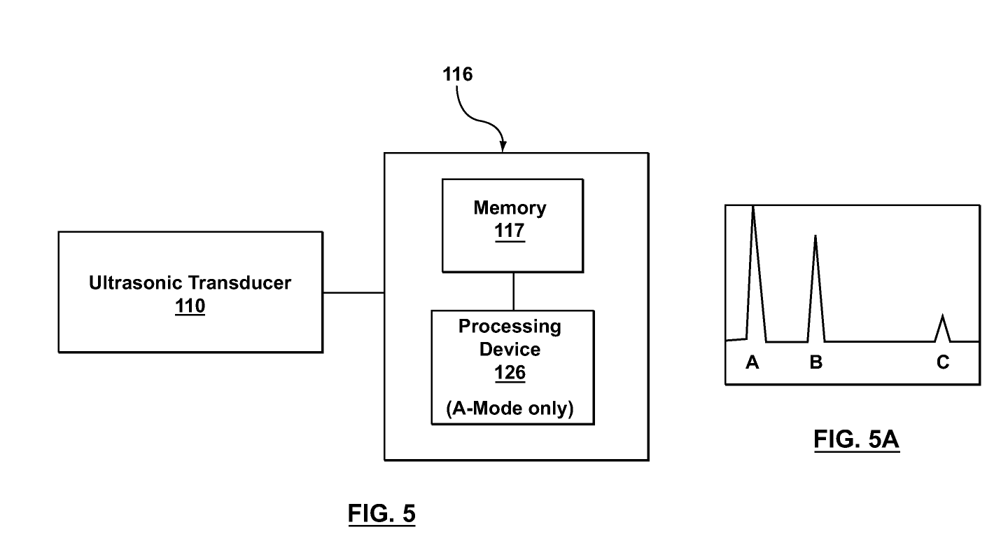

Accordingly to embodiments hereof, ultrasonic transducer 110, processing device 126 and the haptic feedback system having one or more types of feedback function to detect and alert the user of subsurface structures such as luminal or hollow tissue structures or tumor masses located near the tip of surgical tool 110. Luminal structures include for example a ureter, bile duct, bladder, or the like. Referring to the block diagram of FIG. 5, in one embodiment, ultrasonic transducer 110 may operate in A-mode or Beam Reflection Mode. FIG. 5A is a representative diagram of an A-mode sonogram. A-mode is the simplest type of ultrasound in which a single transducer scans a line through the body and echoes are plotted on screen as a function of depth/distance to subsurface structure such as a lumen beneath fat or other obstructive tissue. More particularly, ultrasonic transducer 110 is placed in direct contact with the surface to be investigated and electrical pulses from pulse generator and receiver 116 make ultrasound transducer 110 ring at the desired frequency. The frequency is sufficient to travel through the tissue and can be anywhere between 2 and 10 MHz. The wave travels into the body and comes into focus at a desired depth, which may be manipulated by any method known in the art. Whenever the sound wave encounters a material with a different density (acoustical impedance), part of the sound wave is reflected back to ultrasonic transducer 110 and is detected as an echo. Thus, as the ultrasonic waves travel though tissue and a density change occurs within tissue, a portion of the ultrasonic waves are reflected and a portion of the ultrasonic waves are transmitted. Referring to FIG. 5A, peak or spike A is an echo signal detected by ultrasonic transducer 110 that corresponds to a first density change within tissue, peak or spike B is an echo signal detected by ultrasonic transducer 110 that corresponds to a second density change within tissue, and peak or spike C is an echo signal detected by ultrasonic transducer 110 that corresponds to a third density change within tissue. As the ultrasonic pulse travels through tissue and portions of the sound wave are reflected, less of the sound wave is transmitted resulting in diminishing peaks B and C. For example, in one embodiment, peaks A, B, and C may represent echo signals corresponding to a first proximal surface of an ureter, air in the center of the ureter, and a second or opposing distal surface of the ureter, respectively. When there is a peristaltic pulse in the ureter, peaks A, B, and C will move in accordance with the passage of the pulse as the urine bolus moves past the ultrasound sensor. In a B-Mode configuration, the peristaltic pulse would be observed as a low velocity Doppler signal.

Ultrasound transducer 110 detects the reflected acoustic vibrations or echo signals and generates a signal that is received and processed by pulse generator and receiver 116 in order to determine the distance or depth of subsurface luminal structures. The time it takes for the echo signals to travel back to ultrasound transducer 110 is measured and processed by processing device 126 to calculate the depth of the tissue interface causing the echo. The spikes or peaks of the echo signals, which are associated with depths of different subsurface structures, are identified by a segmentation algorithm and converted into a list of distances or amplitudes. Processing device 126 may also optionally apply standard ultrasound processing to correct for attenuation and other acoustic artifacts to the signal received from ultrasonic transducer 110.

The distance/velocity information from the processed signals is then communicated to the operator as feedback in real-time via one or more output mechanisms on handle 102. In one embodiment having continuos haptic feedback, the distance to the first luminal structure beneath fat or other obstructive tissues is communicated to the operator as an amplitude of a tactile effect via haptic actuator 118. For example, if vibrations are generated as the haptic feedback, the amplitude of the vibrotactile pulse may vary as the tip of surgical tool 100 approaches the luminal structure. Alternatively, the frequency or duration of the vibration may vary as the tip of surgical tool 100 approaches the luminal structure. Other types of haptic feedback may include kinesthetic feedback using solenoids to change the stiffness/damping of handle 102, small air bags that change size in handle 102, or shape changing materials. All embodiments may include combinations of different types of haptic feedback, or combinations of haptic feedback and non-haptic feedback (e.g., audio/visual feedback).

In one embodiment, prior to operation, the user (i.e., a surgeon) may calibrate the feedback alert mechanism by first associating the peaks in the signal with specific structures, including any type of tissue that may be identified by processing device 126 such as luminal or hollow tissue structures or tumor masses. This calibration step provides a template for the processing device to identify similar structures.

In addition to the detection of luminal structures, ultrasonic transducer 110 and haptic feedback system 114 may also function to identify tissue type and detect tissue stiffness when operating in A-mode. When a sound wave encounters a material of a different density and part of the sound wave is reflected back to ultrasonic transducer 110, greater density differences result in stronger/larger echoes. When ultrasound transducer 110 detects the reflected acoustic vibrations or echo signals, the strength of the echo is measured and processed by processing device 126 to determine the identity of the subsurface structure and/or the stiffness of the identified tissue. Processing device 126 then communicates the processed information to one or more of haptic actuator 118, audio device 320, and visual display 422, enabling a surgeon to feel organs for tumors, inflammation, or other anomalies. Examples of tissue that may be identified by processing device 126 include fat/muscle boundaries, tumors or other benign masses, vasculature, muscle-lined luminal structures, and calcification. In one embodiment, an ultrasound contrast agent is administered to the patient preoperatively. The contrast agent significantly increases the impedance mismatch or density differences between tissue layers, thereby resulting in stronger/larger echoes.

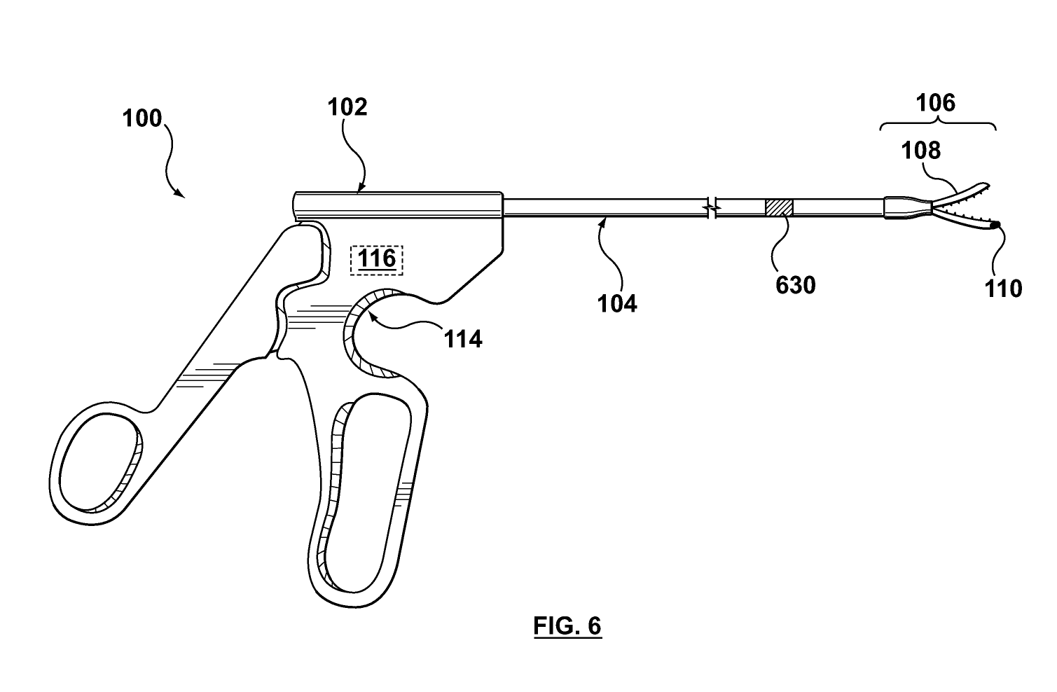

In another embodiment shown in FIG. 6, rather than utilizing ultrasonic transducer 110, a sensor 630 may be coupled to the tip of tool 100 to detect tissue stiffness. Sensor 630 may be a flexible and encapsulating strain gauge. In operation, signals are generated by strain gauge 630 as the tip of tool 100 (e.g., gripper portion) interacts with the bone and various types of tissue found in a human or other animal body and creates deformation in strain gauge 630. The signals received from gauge 630 may be “amplified” by processing device 126 by being converted into corresponding haptic feedback so that the user performing the operation has an “enhanced” feel for the tissue and bone that he/she is navigating through and around. This enhanced feel, a magnification of the forces applied to the surgical tip of the device during use (i.e., cutting, catheterization, etc.) provides the user with better control and sensitivity for using the device effectively, efficiently and with minimal trauma to the patient. Further, during palpation, force sensed at the tool tip is translated into haptic feedback, either to amplify or highlight the internal interaction of the tool tip with the body. Additional examples of sensors that can be coupled to the tool tip include a pressure transducer, a silicon chip that is sensitive to biological materials (i.e., a biosensor), or an electro-magnetic field sensor.

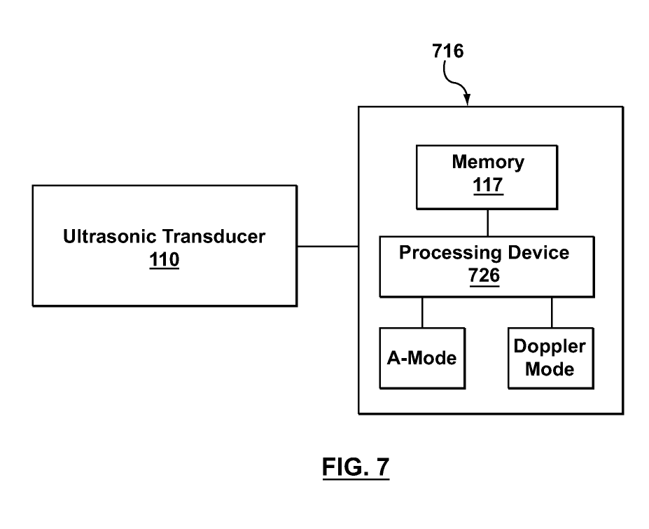

According to embodiments hereof, ultrasonic transducer 110 may operate in one or more additional modes in addition to A-mode. For example, FIG. 7 illustrates an embodiment in which ultrasonic transducer 110 may operate in A-mode or Doppler mode. When ultrasonic transducer 110 operates in Doppler mode, pulse generator and receiver 716 and processing device 726 function to calculate the velocity of subsurface structures. In Doppler mode, processing device 726 measures the direction and speed of moving structures. Such velocity information is useful when identifying vasculature having blood flow there through, such as venous or arterial structures, and for identifying when urine passes through the ureter or the bladder. In one embodiment, the operator may select to receive feedback from ultrasonic transducer 110 in either A-mode or Doppler mode. When the operator desires to switch operational modes, processing device 726 runs different algorithms on the echo signals received from ultrasonic transducer 110. In one embodiment, the operator may select to receive simultaneous feedback from ultrasonic transducer 110 in both modes. Prior to operation, the user (i.e., a surgeon) may select to calibrate the feedback alert mechanism by first associating the peaks in the signal with specific structures, including any type of tissue that may be identified by processing device 126 such as fat/muscle boundaries, tumors or other benign masses, vasculature, muscle-lined luminal structures, and calcification. This calibration step provides a template for the processing device to identify similar structures.

Although FIG. 7 illustrates an embodiment in which ultrasonic transducer 110 may operate in A-mode or Doppler mode, it should be understood by those of ordinary skill in the art that Doppler mode is one of many secondary modes of which ultrasonic transducer 110 may operate. For example, ultrasonic transducer 110 may operate in A-mode or a direct imaging mode. In a direct imaging mode, ultrasonic transducer 110 and the haptic feedback system function to provide feedback to the user regarding density, size, and other characteristics of tissue. Further, it should be understood by those of ordinary skill in the art that ultrasonic transducer 110 may operate in more than two modes. For example, ultrasonic transducer 110 may operate in multiple modes, including A-mode, a direct imaging mode, Doppler mode, B-mode, and/or M-mode. Thus, for all embodiments described herein, ultrasonic transducer 110 may operate in any combination of A-mode, direct imaging mode, Doppler mode, B-mode, and M-mode in order to detect information about subsurface structures, including distance or stiffness information as described above.

The present disclosure describes embodiments that include any type of tools that can be manipulated by an operator. More particularly, the tools described in the present disclosure include a handle portion that mechanically controls a distal portion of the tool. According to embodiments hereof, an ultrasonic transducer at a distal end of a device and a haptic feedback system may collectively function to extract distance, identification information, stiffness, velocity, or other pertinent information regarding subsurface structures that is subsequently communicated to the operator as haptic, audio, and/or visual feedback. Although embodiments disclosed are tools for laparoscopic surgery, other embodiments can be used for non-laparoscopic surgeries such as in vascular or other catheterization where information detected from an ultrasonic transducer on the tool-tip can be communicated back to the catheter handle. Further, for endoscopy procedures, an ultrasonic transducer on a flexible endoscope can communicate local tissue properties. Other embodiments can be used for telesurgery or telepresence in order to, for example, perform routine external examinations by a remote doctor.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A surgical tool comprising:
a handle;
a distal portion connected to the handle via a shaft;
an ultrasonic transducer mounted on the distal portion, the ultrasonic transducer operable in A-mode and configured to detect information about a subsurface structure;
a pulse generator and receiver electrically connected to the ultrasonic transducer, wherein the pulse generator and receiver includes a processing device that receives and processes the subsurface structure information from the ultrasonic transducer; and
a feedback system coupled to the handle and electrically connected to the pulse generator and receiver, wherein the processing device communicates the processed subsurface structure information to the feedback system and the feedback system communicates the processed subsurface structure information to a user.

2. The surgical tool of claim 1, wherein the ultrasonic transducer is forward-looking.

3. The surgical tool of claim 1, wherein the pulse generator and receiver is an external component.

4. The surgical tool of claim 1, wherein the pulse generator and receiver is mounted within the handle.

5. The surgical tool of claim 1, wherein the feedback system includes one or more haptic actuators operable to generate haptic feedback to the handle.

6. The surgical tool of claim 1, wherein the feedback system includes an auditory device.

7. The surgical tool of claim 1, wherein the feedback system includes a visual display.

8. The surgical tool of claim 1, wherein the feedback system is operable to provide feedback in a continuous manner.

9. The surgical tool of claim 1, wherein the feedback system is operable to provide feedback as an alert to notify the user when a particular condition is satisfied.

10. The surgical tool of claim 1, wherein the ultrasonic transducer is operable to detect the distance between the subsurface structure and the ultrasonic transducer by receiving echo signals reflected off the subsurface structure.

11. The surgical tool of claim 1, wherein the ultrasonic transducer is operable to detect the stiffness of the subsurface structure by receiving echo signals reflected off the subsurface structure.

12. The surgical tool of claim 1, wherein the ultrasonic transducer is also operable in Doppler mode to detect velocity information about the subsurface structure.

13. The surgical tool of claim 1, wherein the ultrasonic transducer is also operable in direct imaging mode.

14. The surgical tool of claim 1, wherein the tool is a laparoscopic tool.

15. The surgical tool of claim 1, wherein the tool is an endoscope.

16. The surgical tool of claim 1, wherein the tool is a catheter.

17. A method of providing feedback to a user during a surgical procedure, the method comprising the steps of:
detecting information about a subsurface structure with an ultrasonic transducer mounted on a distal portion of a surgical tool, the ultrasonic transducer operating in A-mode;
processing the detected information; and
communicating the detected information to a user via a feedback system coupled to a handle of the surgical tool.

18. The method of claim 17, wherein communicating the detected information to a user includes providing haptic feedback.

19. The method of claim 17, wherein detecting information about the subsurface structure includes receiving echo signals reflected off the subsurface structure.

20. The method of claim 19, wherein processing the detected information includes detecting the distance between the subsurface structure and the ultrasonic transducer.

21. The method of claim 20, wherein the subsurface structure is a luminal structure.

22. The method of claim 19, wherein processing the detected information includes detecting the stiffness of the subsurface structure.

23. The method of claim 17, wherein the ultrasonic transducer is also operable in Doppler mode to detect velocity information about the subsurface structure.

24. A method of providing feedback to a user during a surgical procedure, the method comprising the steps of:
detecting distance information about a subsurface luminal structure with an ultrasonic transducer mounted on a distal portion of a surgical tool;
processing the detected information to determine the distance between the subsurface luminal structure and the ultrasonic transducer; and
communicating the detected information to a user via a feedback system coupled to a handle of the surgical tool.
